(19) United States
(12) Patent Application Publication
(10) Pub. No.: US 2010/0268196 A1
(43) Pub. Date: Oct. 21, 2010

(54) BRAIDED PEELABLE CATHETER AND METHOD OF MANUFACTURE

(75) Inventors: John Hastings, Savage, MN (US); Alan Twomey, Blaine, MN (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/425,288

(22) Filed: Apr. 16, 2009

(51) Int. Cl. A61M 25/00; B32B 38/04

(52) U.S. Cl. 604/527; 156/256

(57) ABSTRACT

A method of manufacturing a braid-reinforced peelable tubular body is disclosed herein. In one embodiment, the method includes: providing a braided tubular body; forming at least one longitudinally extending slit in tho braided tubular body, resulting in a longitudinally slit braided tubular body, the at least one longitudinally extending slit including slit edges and a severed braid layer of the braided tubular body; placing the longitudinally slit braided tubular body on a mandrel; placing a heat shrink tube about the longitudinally slit braided tubular body; subjecting the heat shrink tube and longitudinally slit braided tubular body to bonding conditions, such as, for example, reflow, laser bonding, thermoforming, etc., thereby causing the slit edges to be joined to each other and resulting in a braid-reinforced peelable tubular body; and removing the braid-reinforced peelable tubular body from the mandrel.

### FIELD OF THE INVENTION

The present invention relates to medical apparatus and methods. More specifically, the present invention relates to tubular delivery devices, such as catheters and sheaths, and methods of using and manufacturing such tubular delivery devices.

### BACKGROUND OF THE INVENTION

Tubular delivery devices, such as catheters and sheaths, are used to deliver implantable medical devices, such as implantable medical leads, to an implantation site within a patient. For example, a catheter or sheath may be routed through the vasculature of the patient such that the distal end of the catheter or sheath is located near the implantation site within the patient's heart. The distal end of the implantable medical lead may then be distally routed through the central lumen of the catheter or sheath to cause the lead distal end to be delivered to the implantation site within the patient. Once the lead distal is properly located at the implantation site within the patient's heart, the tubular delivery device must be removed from about the lead.

A lead connector end on the lead proximal end is used to couple the lead proximal end to an implantable pulse generator, such as a pacemaker or implantable cardioverter defibrillator (“ICD”), which is used to deliver cardio electrotherapy to the implantation site via the lead. Typically, the diameter of the lead connector end exceeds the diameter of the lumen of the tubular delivery device. Thus, to remove the catheter or sheath from about the implanted lead without displacing the lead distal end relative to the implantation site, the tubular body of the catheter or sheath must be longitudinally split. Longitudinal splitting of the tubular body may be accomplished via a slitting tool that slits or cuts the “slittable” tubular body as the tubular body is proximally displaced against the blade of the slitting tool. Alternatively, longitudinal splitting of the tubular body may be accomplished via peeling of the “peelable” tubular body when the tubular body is configured to have a longitudinally extending stress concentration. The stress concentration may be in the form of a longitudinally extending groove formed in the wall of the tubular body or a longitudinally extending strip of material that is different in mechanical properties from the material forming the rest of the tubular wall.

Tubular bodies of catheters and sheaths may be reinforced with braid layers formed of metal or other materials to enhance the mechanical properties (e.g., torqueability, stiffness, kink resistance, pushability, curve retention, etc.) of the tubular bodies. Braid layers may be employed in tubular bodies and still result in tubular bodies that are slittable because the slitting tool is capable of slitting such braid-reinforced tubular bodies. However, this has not been the case with peelable tubular bodies. Specifically, heretofore, no tubular body for a catheter or sheath has been available that is both braid-reinforced and peelable because the presence of a braid layer made the tubular body incapable of being peeled.

Many physicians prefer the peeling process over the slitting process because the peeling process offers more simplicity and control compared to the slitting process and does not require a separate tool. However, because peelable tubular bodies have heretofore lacked the ability to be braid-reinforced and, therefore, lacked the mechanical properties (torqueability, stiffness, kink resistance, pushability, curve retention, etc.) of a braid-reinforced slittable tubular body, slittable catheters and sheaths have historically outsold peelable catheters and sheaths by large amounts (e.g., three to one).

There is a need in the art for a catheter or sheath having a braid-reinforced tubular body that is peelable and still offers mechanical characteristics similar to braid-reinforced tubular bodies known in the art. There is also a need in the art for methods of manufacturing and using such a peelable, braid-reinforced tubular body for catheter or sheath.

### BRIEF SUMMARY OF THE INVENTION

A method of manufacturing a braid-reinforced peelable tubular body is disclosed herein. In one embodiment, the method includes: provide a braided tubular body; form at least one longitudinally extending slit in the braided tubular body, resulting in a longitudinally slit braided tubular body, the at least one longitudinally extending slit including slit edges and severing a braid layer of the braided tubular body; place the longitudinally slit braided tubular body on a mandrel; place a heat shrink tube about the longitudinally slit braided tubular body; subject the heat shrink tube and longitudinally slit braided tubular body to bonding conditions (e.g., reflow, laser bonding, thermoforming, etc.), thereby causing the slit edges to be joined to each other and resulting in a braid-reinforced peelable tubular body; and remove the braid-reinforced peelable tubular body from the mandrel.

A braid-reinforced peelable tubular body manufactured according to the above-mentioned method is also disclosed herein.

A catheter or sheath is also disclosed herein. In one embodiment, the catheter or sheath may include a braid-reinforced peelable tubular body having a wall with a circumference. The wall may include a braid layer and at least one longitudinally extending stress concentration. The braid layer may extend uninterrupted along the circumference except in a longitudinally extending region of the stress concentration.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of a catheter or sheath having a braided peelable tubular body.

FIG. 2 is a transverse cross section of the braided tubular body as taken along section line 2-2 in FIG. 1.

FIG. 3 is a longitudinal side view of a portion of the braided tubular body, wherein various layers of the tubular body are removed in some locations to reveal layers or structure below that would otherwise be hidden from view.

FIG. 4 is a flow diagram illustrating three embodiments of a method of manufacturing the braided peelable tubular body.

FIG. 5 is an isometric of a traditional braided tubular body that has been slit in preparation for manufacturing the braid-reinforced tubular body depicted in FIGS. 1-3.

FIG. 6 is a cross section of the braid-reinforced tubular body halves assembled onto a reflow mandrel.

FIG. 7 is the same view as FIG. 6, except of another embodiment.

FIG. 8 is the same view as FIG. 6, except of yet another embodiment.

### DETAILED DESCRIPTION

A tubular delivery device 10, such as, for example, a catheter or sheath 10, is disclosed herein. The catheter or sheath 10 may include a braided or braid-reinforced peelable tubular body 12. The catheter or sheath 10 may also include a splittable hub 14 coupled to a proximal end 16 of the braid-reinforced peelable tubular body 12. The hub 14 may facilitate a hemostasis valve or other device to be coupled to the proximal end 16 of the tubular body 12. The catheter or sheath 10 advantageously provides the mechanical characteristics of a braided tubular body while being readily peelable.

The following description presents preferred embodiments of the braid-reinforced peelable tubular body 12 and its method of manufacture and represents the best mode contemplated for practicing the braid-reinforced peelable tubular body 12 and its method of manufacture. This description is not to be taken in a limiting sense, but is made merely for the purpose of describing the general principles of the braid-reinforced peelable tubular body 12 and its method of manufacture, the scope of both being defined by the appended claims.

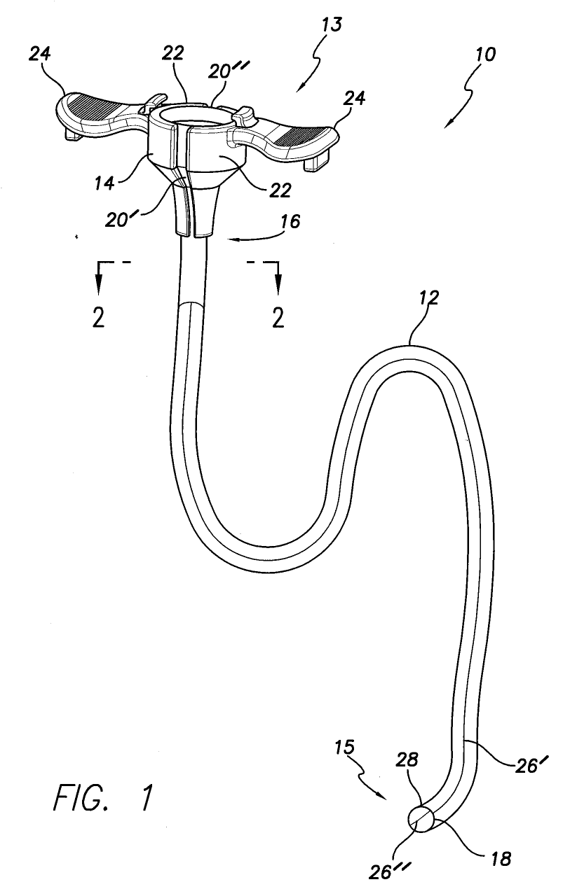

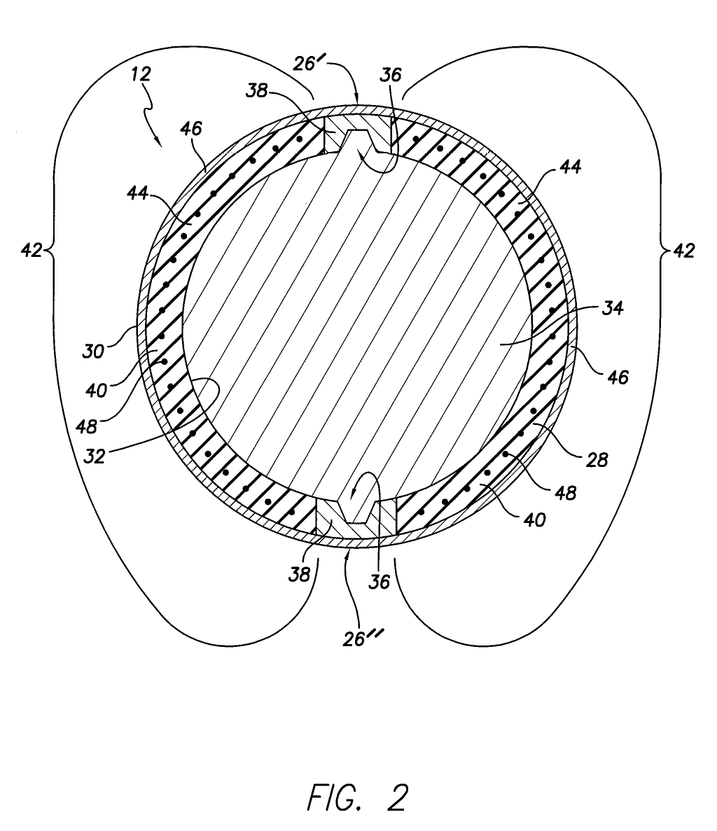

For a detailed discussion regarding the braid-reinforced catheter or sheath 10, reference is made to FIGS. 1 and 2. FIG. 1 is an isometric view of an embodiment of the catheter or sheath 10 employing the braid-reinforced peelable tubular body 12, and FIG. 2 is a transverse cross section of the braid-reinforced tubular body 12 of the catheter or sheath 10 as taken along section line 2-2 in FIG. 1. As indicated in FIG. 1, the catheter or sheath 10 may include a braided or braid-reinforced peelable tubular body 12, a proximal end 13, a splittable hub 14 at the proximal end 13, and a distal end 15. The tubular body 12 may include a proximal end 16 and a distal end 18.

The hub 14 may be employed to couple a hemostasis valve or other medical device to the proximal end 13 of the catheter or sheath 10. The hub 14 may be longitudinally splittable via the presence of a longitudinally extending stress concentration 20 defined in the wall 22 of the hub 14. The hub wall stress concentration 20 may be in the form of a splitting groove defined in the hub wall 22. As can be understood from FIG. 1, the hub wall 22 may have two longitudinally extending stress concentrations 20′, 20″ defined in the wall 22 at opposite locations from each other in the circumference of the wall 22. Thus, the hub wings 24 may be grasped and forced apart to cause the hub 14 to split into two generally equal halves on account of the two oppositely located stress concentrations 20′, 20″. In other embodiments, the hub 14 may have a greater or lesser number of stress concentrations 20.

As shown in FIGS. 1 and 2, the tubular body 12 may include two longitudinally extending stress concentrations 26′, 26″ formed in the wall 28 of the tubular body 12. The wall 28 defines an outer circumferential surface 30 of the tubular body 12 and an inner circumferential surface 32 of the tubular body 12. The inner circumferential surface 32 may define a central lumen 34 of the tubular body 12.

Similar to the stress concentrations 20′, 20″ of the hub 14, the stress concentrations 26′, 26″ of the tubular body 12 may be formed in the wall 28 of the tubular body 12 at opposite locations from each other in the circumference of the wall 28. These oppositely located tubular body stress concentrations 26′, 26″ may be generally aligned with the hub stress concentrations 20′, 20″ such that the splitting of the hub 14 may be used to peel the tubular body 12 into two generally equal halves. In other embodiments, the tubular body 12 may have a greater or lesser number of stress concentrations 26.

As indicated in FIG. 2, in one embodiment, the stress concentrations 26′, 26″ may be formed by a groove 36 defined in the inner circumferential surface 32 of the tubular body 12 and extending the length of the stress concentrations 26′, 26″. In other embodiments, the groove 36 may be defined in the outer circumferential surface 30 or in both the inner and outer circumferential surfaces 32, 30.

As can be understood from FIG. 2, the stress concentrations 26′, 26″ may be formed of a material 38 or have a makeup or configuration that is mechanically dissimilar from the mechanical characteristics of the material 40 or makeup or configuration that may form the majority of the non-stress concentration portions 42 of the wall 28. In some embodiments, the wall 28 may include an inner layer 44 and an outer layer 46 extending about the inner layer 44. In such an embodiment, the stress concentrations 26′, 26″ and the outer layer 46 may be formed of a first type of polymer material (e.g., polyether block amide (“PEBAX”), nylon, polyurethane, etc.), while the inner layer 44 may be formed of second type of polymer material (e.g., PEBAX (preferably of a durometer higher than the PEBAX of the outer layer), nylon, polyurethane, polytetrafluoroethylene (“PTFE”), fluorinated ethylene propylene (“FEP”), etc.) different from the first type of polymer material and including a braid layer 48 embedded therein.

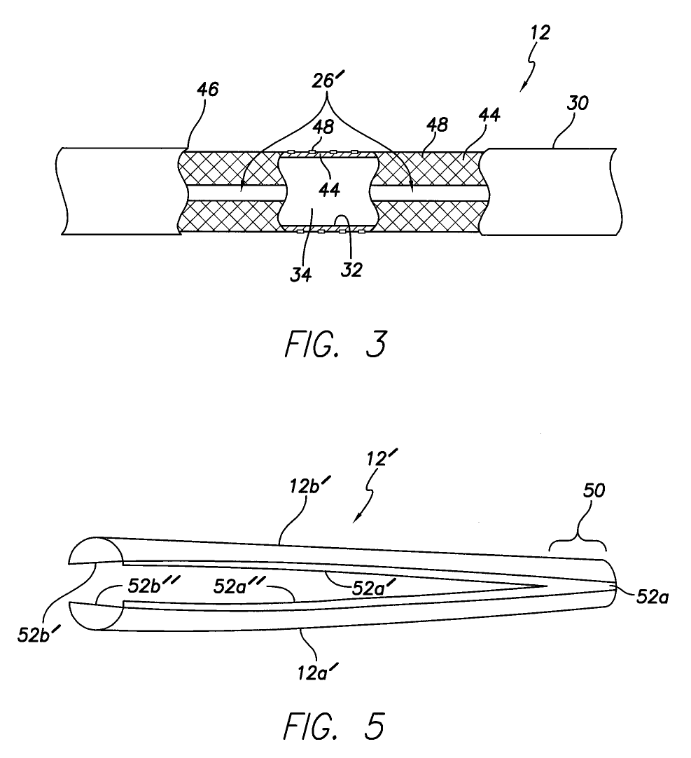

Further understanding regarding the configurations of the braid layer 48 and stress concentrations 26 of the braid-reinforced peelable tubular body 12 of FIGS. 1 and 2 may obtained from FIG. 3, which is a longitudinal side view of a portion of the braid-reinforced tubular body 12, wherein various layers of the tubular body 12 are removed in some locations to reveal layers or structure below that would otherwise be hidden from view. As shown in FIG. 3, the outer layer 46 may extend over the braid layer 48, which may extend over the inner layer 44, the braid layer 48 being embedded in the outer layer 46. In another embodiment, as depicted in FIG. 2, the outer layer 46 may extend over the inner layer 44, which contains the braid layer 48 embedded therein. Regardless of which layer the braid 48 is embedded in, as can be understood from FIGS. 2 and 3, the tubular body 12 is braid-reinforced throughout its entire circumferential extent, except along the length of the stress concentrations 26′, 26″. The lack of braid layer 48 in the vicinity of the stress concentrations 28′, 28″ enables the tubular body 12 of the catheter or sheath 10 to be peeled in a fashion identical to a traditional peelable catheter while still offering mechanical properties very similar to those of a traditional braided catheter due to the presence of the braid layer 48 in all other areas of the tubular body 12.

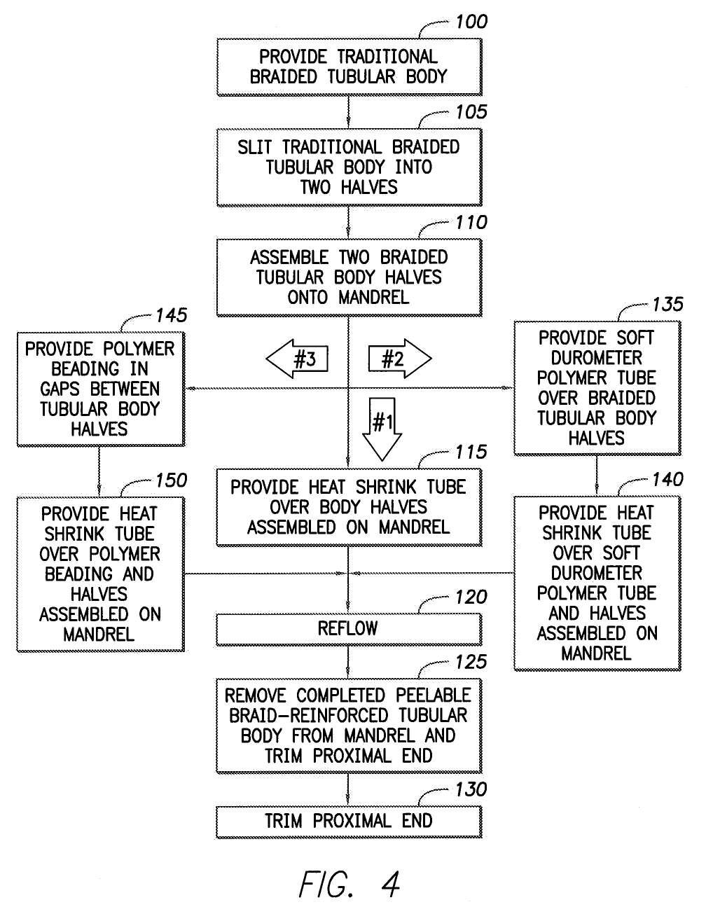

For a discussion regarding a first embodiment of a method of manufacturing the braid-reinforced peelable tubular body 12, reference is first made to FIGS. 4 and 5. FIG. 4 is a flow diagram illustrating three embodiments of the manufacturing method, and FIG. 5 is an isometric of a traditional braided tubular body 12′ that has been slit in preparation for manufacturing the braid-reinforced tubular body 12 described above.

A traditional braided tubular body 12′ is provided, wherein the braid layer of the traditional braided tubular body 12′ is circumferentially continuous [block 100 of FIG. 4]. Such a traditional braided tubular body 12′ may be constructed from a two-process extrusion, reflow, or any other commonly used tubular body manufacturing processes.

As can be understood from FIG. 5, the traditional braided tubular body 12′ may be longitudinally slit into two halves 12a′, 12b′ along its entire length, with the exception of a most proximal segment 50 of the tubular body 12′ having a length of approximately one inch, the most proximal segment 50 remaining un-slit [block 105 of FIG. 4]. The most proximal segment 50 may remain un-slit to aid in handling. As indicated in FIG. 5, the tubular body 12′, on account of the manufacturing processes used to manufacture the traditional braided tubular body 12′, may have two thin strips 52a, 52b constructed of softer material as compared to the material adjacent the strips 52a, 52b in the traditional braided tubular body 12′. The slitting process may be accomplished using a simple blade fixture, laser, or other cutting mechanism common to tubular body manufacturing.

When the traditional tubular body 12′ is slit according to [block 105] of FIG. 4, the traditional tubular body 12′ may be slit along these strips 52a, 52b to form corresponding strip edges 52a′, 52a″ and 52b′, 52b″, as depicted in FIG. 5. These strip edges 52a′, 52a″ and 52b′, 52b″, which may extend the entire length of the slit traditional tubular body 12′, may be used to surround and form the score features 26′, 26″ of the peelable braid-reinforced tubular body 12 described above with respect to FIGS. 1-3.

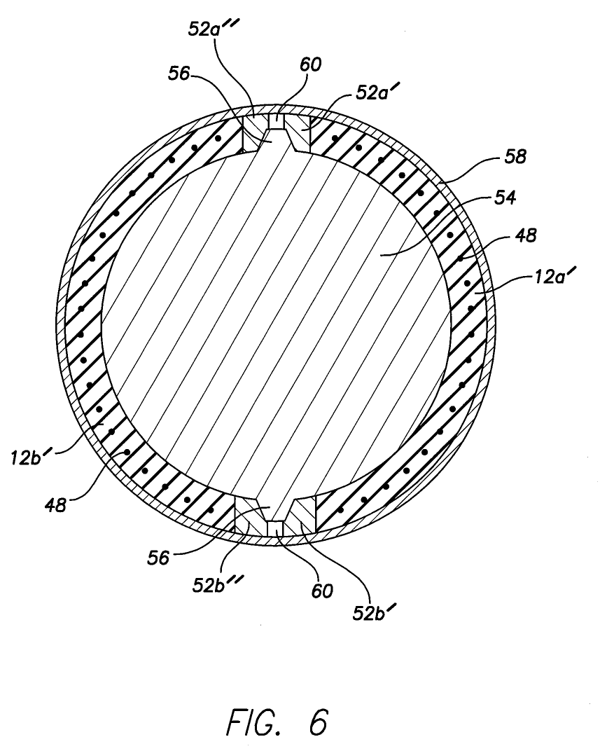

As shown in FIG. 6, which is a cross section of the tubular body halves 12a′, 12b′, the braid-reinforced tubular body halves 12a′, 12b′ are assembled onto a reflow mandrel 54 [block 110 of FIG. 4]. The mandrel 54 may include protruding geometry 56 to form score lines. A shrink tube 58 formed of FEP or other shrink tube material may be pulled or otherwise provided about the outer circumferential surface of the braid-reinforced layer 44 provided by the tubular body halves 12a′, 12b′ [block 115 of FIG. 4]. When tubular body halves 12a′, 12b′ and heat shrink tube 58 are assembled on the mandrel 54 as indicated in FIG. 6, gaps 60 may exist between the strip edges 52a′, 52a″ and 52b′, 52b″. The assembly depicted in FIG. 6 may be subjected to a reflow process [block 120 of FIG. 4]. In other words, the assembly depicted in FIG. 6 is subjected to bonding conditions (e.g., reflow, laser bonding, thermoforming, etc.) that cause the strip edges 52a′, 52a″ and 52b′, 52b″ to flow into the gaps 60, filling the gaps 60 and forming the stress concentration lines 26′, 26″ that join the tubular body halves 12a′, 12b′ into a braid-reinforced tubular body 12 that is similar to that of FIG. 1-3, less the outer layer 46. The protruding geometry 56 of the mandrel 54 forms the score lines 36 in the interior surface 32 in the vicinity of the stress concentrations 26′, 26″. Once the reflow process is completed, the material forming the shrink tube 58 may be removed from about the completed tubular body 12. The completed peelable braid-reinforced tubular body 12 that is similar to that of FIGS. 1-3, less the outer layer 46, may be removed from the mandrel 54 [block 125 of FIG. 4]. The approximately one inch long non-slit portion 50 discussed above with respect to FIG. 5 (i.e., the portion 50 of the braid-reinforced tubular body 12′ not slit in [block 105 of FIG. 4]) may be cut from the completed peelable braid-reinforced tubular body 12 [block 130 of FIG. 4].

As can be understood from the process described above with respect to FIGS. 1-6, the reflow performed with the heat shrink tube 58 serves the purpose of re-forming the tubular body 12′, which was slit in [block 105 of FIG. 4]. During the reflow process, the tubular body 12′ re-assumes its original shape. However the score sections 26′, 26″ remain free of braid 48 due to the original slit process, thereby resulting in a peelable braid-reinforced tubular body 12 similar to that depicted in FIGS. 1-3.

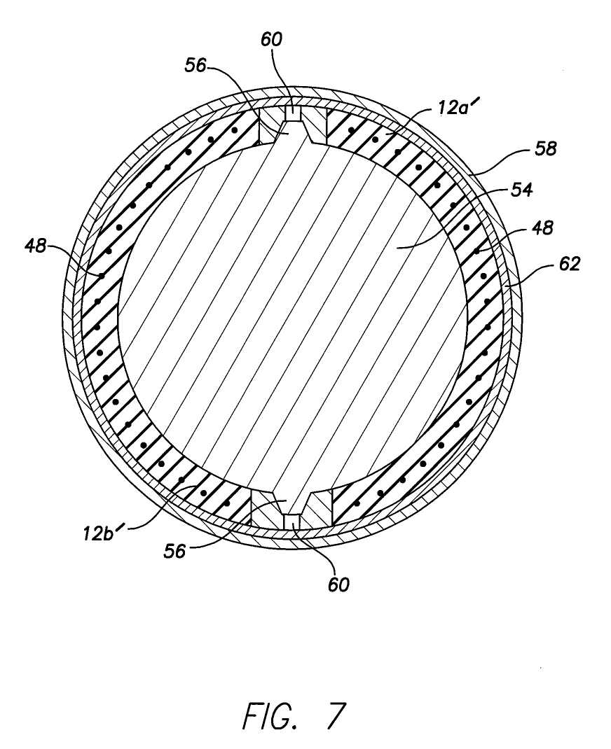

For a discussion of a second manufacturing embodiment, reference is made to FIG. 7, which is the same view as FIG. 6, except of the second manufacturing embodiment. In the second manufacturing embodiment, prior to the placement of the heat shrink tube 58 about the outer surfaces of the tubular body halves 12a′, 12b′ and, wherein the tubular body halves 12a′, 12b′ may not have any or sufficient strip edges 52a′, 52a″ and 52b′, 52b″ to fill in the gaps 60, a soft durometer polymer tube 62 may be placed about the outer circumferential surfaces of the tubular body halves 12a′, 12b′ [block 135 of FIG. 4]. The soft durometer polymer tube 62 may be formed of the same material as what the strip edges 52a′, 52a″ and 52b′, 52b″ would have been made of, for example, soft durometer PEBAX, polyurethane, nylon, etc. The heat shrink tube 58 may be pulled over the soft durometer polymer tube 62 [block 140 of FIG. 4]. The assembly depicted in FIG. 7 may be subjected to the bonding conditions or reflow process [block 120 of FIG. 4]. Once the reflow process is completed, the material forming the shrink tube 58 may be removed from about the completed tubular body 12. The completed peelable braid-reinforced tubular body 12 may be removed from the mandrel 54 [block 130 of FIG. 4]. The non-slit end 50 may then be trimmed from the complete peelable braid-reinforced tubular body 12 [block 135 of FIG. 4]. The resulting peelable braid-reinforced tubular body 12 may have the configuration depicted in FIG. 2, wherein the soft durometer polymer tube 62 forms the outer layer 46 and the stress concentration lines 26′, 26″ that join the tubular body halves 12a′, 12b′ into the braid-reinforced tubular body 12 of FIG. 1-3, and the braided halves 12a′, 12b′ form the inner layer 44.

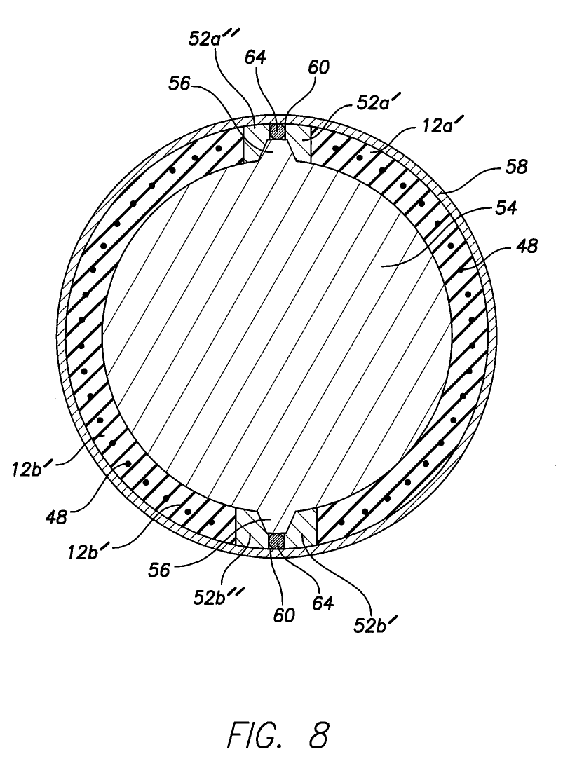

For a discussion of a third manufacturing embodiment, reference is made to FIG. 8, which is the same view as FIG. 6, except of the third manufacturing embodiment. In the third manufacturing embodiment, prior to the placement of the heat shrink tube 58 about the outer surfaces of the tubular body halves 12a′, 12b′, a polymer beading 64 may be placed in each of the gaps 60 between the tubular body halves 12a′, 12b′ [block 145 of FIG. 4]. In a first version of embodiment three, the polymer beading 60 may be provided where the tubular body halves 12a′, 12b′ may not have any or sufficient strip edges 52a′, 52a″ and 52b′, 52b″ to fill in the gaps 60. In a second version of embodiment three, the polymer beading 60 may be provided despite the tubular body halves 12a′, 12b′ having sufficient strip edges 52a′, 52a″ and 52b′, 52b″ to fill in the gaps 60. In the second version of embodiment three, the strip edges 52a′, 52a″ and 52b′, 52b″ may be made of, for example, PEBAX, nylon, polyurethane, etc., and the polymer beading 64 may be made of another material such as PTFE, FEP, etc. The difference in materials between the strip edges 52a′, 52a″ and 52b′, 52b″ and the polymer beading 64 may enhance the resulting stress concentrations and the peelability of the resulting braid-reinforced peelable tubular body 12.

The heat shrink tube 58 may be pulled over the polymer beading 64 and tubular body halves 12a′, 12b′ [block 150 of FIG. 4]. The assembly depicted in FIG. 8 may be subjected to the bonding conditions or reflow process [block 120 of FIG. 4]. Once the reflow process is completed, the material forming the shrink tube 58 may be removed from about the completed tubular body 12. The completed peelable braid-reinforced tubular body 12 may be removed from the mandrel 54 [block 130 of FIG. 4]. The non-slit end 50 may then be trimmed from the completed peelable braid-reinforced tubular body 12 [block 135 of FIG. 4]. The resulting peelable braid-reinforced tubular body 12 may have a configuration similar to that depicted in FIG. 2, less the outer layer 46. In other words, the polymer beading 64 forms the stress concentration lines 26′, 26″ that join the tubular body halves 12a′, 12b′ into a braid-reinforced tubular body 12 similar to that of FIG. 1-3, less the outer layer 46, and the braided halves 12a′, 12b′ form the inner layer 44.

The embodiments depicted in FIGS. 1-8 depict tubular bodies 12 with two stress concentration lines 26′, 26″ and score lines 36 located at 180 degrees from each other about the circumference of the tubular bodies 12. However, in other embodiments, the tubular bodies 12 may have more than or less than two stress concentration lines 26′, 26″ and score lines 36, and such peel enabling features may be spaced apart from each other by spacings other than 180 degrees.

Although the present invention has been described with reference to preferred embodiments, persons skilled in the art will recognize that changes may be made in form and detail without departing from the spirit and scope of the invention.

What is claimed is:

1. A method of manufacturing a braid-reinforced peelable tubular body, the method comprising:
providing a braided tubular body;
forming at least one longitudinally extending slit in the braided tubular body, resulting in a longitudinally slit braided tubular body, the at least one longitudinally extending slit including slit edges and a severed braid layer of the braided tubular body;
placing the longitudinally slit braided tubular body on a mandrel;
subjecting the longitudinally slit braided tubular body to bonding conditions, thereby causing the slit edges to be joined to each other and resulting in a braid-reinforced peelable tubular body; and
removing the braid-reinforced peelable tubular body from the mandrel.

2. A catheter or sheath comprising a braid reinforced peelable tubular body manufactured according to the method of claim 1.

3. The method of claim 1, further comprising placing a heat shrink tube about the longitudinally slit braided tubular body prior to subjecting the longitudinally slit braided tubular body to the bonding conditions.

4. The method of claim 1, wherein the bonding conditions include at least one of reflow, laser bonding, and thermoforming.

5. The method of claim 1, wherein the at least one longitudinally extending slit includes two such slits.

6. The method of claim 5, wherein the two such slits are located approximately 180 degrees apart from each other about the circumference of the longitudinally slit braided tubular body.

7. The method of claim 1, wherein the slit edges includes a soft durometer polymer, and the bonding conditions cause the soft durometer polymer to reflow, resulting in the slit edges joining each other.

8. A catheter or sheath comprising a braid-reinforced peelable tubular body manufactured according to the method of claim 7.

9. The method of claim 7, further comprising providing a polymer beading between the slit edges prior to the subjecting of the longitudinally slit braided tubular body to the bonding conditions.

10. The method of claim 9, wherein the polymer beading and soft durometer polymer of the slit edges are caused to join via the bonding conditions.

11. A catheter or sheath comprising a braid-reinforced peelable tubular body manufactured according to the method of claim 10.

12. The method of claim 10, wherein the polymer beading includes PTFE.

13. The method of claim 7, wherein the resulting joined together slit edges form a stress concentration that facilitates the resulting braid-reinforced peelable tubular body being peeled along the stress concentration.

14. The method of claim 13, wherein the mandrel includes a feature that positionally coincides with the location of the slit edges on the mandrel to create score lines in the resulting braid-reinforced peelable tubular body in the vicinity of the stress concentration.

15. The method of claim 13, wherein the braid-reinforced peelable tubular body is at least one of a sheath or catheter.

16. The method of claim 3, further comprising placing a soft durometer polymer tube about the longitudinally slit braided tubular body, the soft durometer polymer tube being located between the longitudinally slit braided tubular body and the heat shrink tube.

17. The method of claim 16, wherein the bonding conditions cause the soft durometer polymer tube to join the slit edges to each other.

18. A catheter or sheath comprising a braid-reinforced peelable tubular body manufactured according to the method of claim 17.

19. The method of claim 17, wherein the bonding conditions further cause the soft durometer polymer tube to form an outer layer about the resulting braid-reinforced peelable tubular body.

20. The method of claim 19, wherein the resulting joined together slit edges form a stress concentration that facilitates the resulting braid-reinforced peelable tubular body being peeled along the stress concentration.

21. The method of claim 20, wherein the mandrel includes a feature that positionally coincides with the location of the slit edges on the mandrel to create score lines in the resulting braid-reinforced peelable tubular body in the vicinity of the stress concentration.

22. The method of claim 17, wherein the braid-reinforced peelable tubular body is at least one of a sheath or catheter.

23. The method of claim 3, further comprising placing a polymer beading between the slit edges, the heat shrink tube being located about the polymer beading and the longitudinally slit braided tubular body.

24. The method of claim 23, wherein the bonding conditions cause the polymer beading to join the slit edges to each other.

25. The method of claim 24, wherein the resulting joined together slit edges form a stress concentration that facilitates the resulting braid-reinforced peelable tubular body being peeled along the stress concentration.

26. The method of claim 25, wherein the mandrel includes a feature that positionally coincides with the location of the slit edges on the mandrel to create score lines in the resulting braid-reinforced peelable tubular body in the vicinity of the stress concentration.

27. The method of claim 24, wherein the braid-reinforced peelable tubular body is at least one of a sheath or catheter.

28. A catheter or sheath comprising a braid-reinforced peelable tubular body manufactured according to the method of claim 24.

29. A catheter or sheath comprising a braid-reinforced peelable tubular body including a wall including a circumference, the wall including a braid layer and at least one longitudinally extending stress concentration, the braid layer extending uninterrupted along the circumference except in a longitudinally extending region of the stress concentration.

30. The catheter or sheath of claim 29, wherein the stress concentration includes a polymer strip including a material different from a material in which the braid layer is imbedded.

31. The catheter or sheath of claim 30, wherein the stress concentration includes a score line defined in a circumferential surface of the tubular body.
